(19) United States
(12) Patent Application Publication
(10) Pub. No.: US 2012/0088225 A1
(43) Pub. Date: Apr. 12, 2012

(54) Method of Detecting Major In-Hospital Ischemic Complications and Predicting Length of Stay at an Intensive Care Unit

(76) Inventors: Jan LISKA, Stockholm (SE); Anders Franco-Cereceda, Stockholm (SE); Måns Alfvén, Stockholm (SE)

(21) Appl. No.: 13/267,940

(22) Filed: Oct. 7, 2011

Related U.S. Application Data
(60) Provisional application No. 61/391,293, filed on Oct. 8, 2010.

(51) Int. Cl. C12Q 1/25

(52) U.S. Cl. 435/4

(57) ABSTRACT

Methods of detecting major ischemic complications in critically ill patients comprise a) determining the blood concentration of glycerol at least once per hour; b) comparing the obtained blood concentration of glycerol with a defined critical level; and c) based on the comparison of step b), identifying patients experiencing or being at risk for major ischemic complications in order to select such patients for intensified surveillance and treatment. The methods allow early and reliable detection of major ischemic complications and identification of patients at risk for prolonged stay at an intensive care unit. The patients preferably have undergone surgical intervention with a following reperfusion or can be patients suffering from the complications of a trauma at an intensive care unit, or have undergone cardiac surgery.

### RELATED APPLICATION

The present application claims priority under 35 U.S.C. 119 of U.S. application No. 61/391,293 filed Oct. 8, 2010.

### FIELD OF INVENTION

The present invention relates to methods of using monitored blood concentration of glycerol as a diagnostic and prognostic marker in critically ill patients.

### BACKGROUND OF THE INVENTION

Major ischemic complications, including peri-operative myocardial infraction, strokes/TIA, splanchnic ischemia, limb ischemia and multi-organ failure, are a significant cause of morbidity and mortality in post-surgical and other critically ill patients.

Peri-operative Myocardial Infarction (PMI, defined to include intra- and post-operative myocardial infarction) is a serious complication after surgery. It is a major cause of morbidity and mortality. It has also been associated with a significant increase in intensive care unit time, hospital length of stay, and overall hospital costs. Reported incidence rates in e.g. cardiac surgery vary from 3% to 21%. This broad variation is primarily the result of differences in diagnostic criteria Immediate detection of peri-operative ischemia is essential for effective salvage intervention. Current methods for detection all have significant limitations.

Stroke is another serious ischemic complication occurring in 3% to 9% of patients after cardiac surgery. Patients with post-operative strokes have up to a 10-fold increase in mortality and a 3-fold increase in length of hospitalization. Detection is often not possible until clinical signs of neurologic deficiency are shown and is often not confirmed until a Computerized Tomography (CT) or Magnetic Resonance Imaging (MRI) have been performed.

Lactate measurement in critically ill patients has traditionally been used as a marker of hypoxia and ischemia and to stratify patients with poor outcome. The normal reference values for the blood concentration of lactate are traditionally considered 1±0.5 mmol/l in normal patients and >2 mmol/l in critically ill patients. Thus, approximately 2 mmol/l can be defined as a critical level of lactate. However, increased lactate levels in critically ill patients are generally unspecific and increased levels are common without the presence of major ischemic complications.

Glycerol is suggested as a marker for ischemia. In J Neurol Neursurg Psychiatry, 1998; 64; 486-491, L Hillered et al describes sampling of glycerol by microdialysis from brain interstitial fluid in order to evaluate its potential as a marker for membrane phospholipid degradation in acute brain injury. Glycerol is found correlate well with the ratio lacate/puruvate as an ischemic marker (glycerol is measured enzymatically). Furthermore, in J Neurol Neurosurg Psychiatry 2001; 71:455-461, P Frykholm et al concluded that a sustained increase in interstitial glycerol was indicative of severe ischemia and that interstitial glycerol in combination with other biochemical markers such as the lactate/pyruvate-ratio and glutamate may be useful for clinical monitoring of the ischemic brain.

In an early study published in British Heart Journal, 1971; 33; 848-888, S Carlstrom et al determined plasma glycerol concentration in patients admitted to hospital due to chest pain.

Patients having myocardial infarction complicated by arrhythmias showed significantly higher plasma glycerol concentrations compared to patients with uncomplicated infarction or angina.

In another study by T Backstrom et al published in Scandinavian Cardiovascular Journal, 2000; 36(1); 27-34 glycerol is monitored in an animal model by means of microdialysis in the myocardial venous outflow during ischemia and reperfusion. It was observed that glycerol was progressively released during ischemia and that myocardial infarction resulted in additional release of glycerol in the early reperfusion.

Even if glycerol has been suggested as an ischemic marker, it has not been been studied to a larger extent in post-surgical and other critically ill patients and it's relevance as a diagnostic and prognostic marker compared to conventional ischemic markers like lactate is yet to be fully exploited.

### DESCRIPTION OF THE INVENTION

In general terms the present invention relates to methods of monitoring the blood concentration of glycerol in post-surgical and other critically ill patients in order to early and reliably detect major ischemic complications and to identify patients at risk for prolonged stay at an intensive care unit. The patients to be subjected to the methods of the invention preferably have undergone surgical intervention with a following reperfusion or can be patients suffering from the complications of a trauma at an intensive care unit. An especially preferred group of patients have undergone cardiac surgery.

The inventive methods involve the steps of determining the blood concentration of glycerol at least once per hour; comparing the so obtained data of the blood concentration of glycerol with a defined critical level of the blood concentration of glycerol; and based on the comparison between the obtained data and the critical level identify patients experiencing or being at risk for major ischemic complications and subsequently direct treatment for these patients. Based on the comparison with the predetermined critical glycerol level, the method is further useful for predicting length of stay at an intensive care unit.

The identification of patients at risk for major ischemic complications is performed when the patient's blood concentration of glycerol at least one time has exceeded the defined critical level of glycerol. Alternatively, identification of such patients is performed when there has been a specific time period during which the patient's blood concentration of glycerol has exceeded the defined critical level of glycerol. Typically, such a specific time period is at least one hour.

In the context of the present invention, a defined critical level for the blood concentration of glycerol is estimated to be approximately 350 μmol/l to 400 μmol/l. A normal value for the blood concentration of glycerol is defined to be approximately between 20 μmol/l and 270 μmol/l (McCabe ERB: Disorders of glycerol metabolism. The Metabolic and Molecular Bases of Inherited Disease. 8^{th} Edition. C. R. Scriver, A. L. Beaudet, W. S. Sly, D. Valle, B. Childs and B. Vogelstein, editors, New York: McGraw-Hill, 2001).

Also in the context of the present invention, in patients having undergone cardiac or other major surgery, it is preferable to start determining the blood concentration of glycerol at a certain time after the patient has been subjected to reperfusion after the surgical intervention. This time period is preferably sufficiently extended to clear the patient or organ from glycerol accumulated as a consequence of the surgery. A suitable such clearance time period is estimated to be approximately 30 minutes. After such clearance period, obtaining data of the blood concentration of glycerol is typically performed during at least four hours. The analytical method of determining the blood concentration of glycerol is not critical for performing the inventive method. The skilled person is aware of several suitable methods including enzymatic methods.

Also, whether the concentration of whole blood glycerol or plasma glycerol is used is not critical for performing the inventive method. In the context of the inventive method the concentration of blood glycerol and plasma glycerol are interchangeable.

It is another aspect of the invention that sampling of the blood for analysis can be performed from systemic blood such as arterial or central venous sampling, as well as samples from the Coronary Sinus (CS), the venous outflow of the heart. In the following exemplifying part a microdialysis (MD) technology for sampling from the CS is demonstrated, however, the invention can be performed by means of sampling with a number of different technologies securing representative blood samples.

In one embodiment of the invention, the methods further comprises determining the blood concentration of lactate, preferably the systemic blood concentration of lactate, in conjunction with determining the blood concentration of glycerol. Combining determination of the blood concentration of glycerol and lactate can help differentiate between patients with major ischemic complications and other types of complications and patients without any complications. Patients with increased blood concentration of lactate, often above the previously mentioned level of 2 mmol/l, but with a blood concentration of glycerol below the defined critical level, are likely to have a better clinical outcome and shorter stay at an intensive care unit compared to patients with both increased blood concentration of lactate and a blood concentration of glycerol above the defined critical level. The critical level of glycerol is estimated to be approximately 350 μmol/l to 400 μmol/l

The invention further embodies a method of detecting major ischemic complications, in post-surgical or other critically ill patients, wherein the blood concentration of lactate of the patient is measured. The method further includes detection of an increase in the blood concentration of lactate, preferably compared to a critical level of lactate such as the previously mentioned level of 2 mmol/l, and if an increase in the blood concentration of lactate is determined, then determining the blood concentration of glycerol and comparing the so obtained data of the blood concentration of glycerol with a defined critical level of the blood concentration of glycerol. Based on the comparison patients are identified who are experiencing or being at risk for major ischemic complications so such patients are selected for intensified surveillance and treatment. As previously outlined, the method is useful for predicting the length of stay at an intensive care unit and the critical level of glycerol is estimated to be approximately 350 mol/l to 400 μmol/l. For post-surgical patients, such as cardiac surgery patients, the method preferably involves obtaining data of the blood concentration of lactate during at least four hours following reperfusion. The lactate detection is preferably performed by continuous measurements in systemic blood, but other methods are conceivable to the skilled person.

### BRIEF DESCRIPTION OF THE DRAWINGS

The following detailed description will be more fully understood when viewed together with the drawings, in which:

FIG. 1 is a table demonstrating the clinical outcome per patient having significantly elevated values for the blood concentration of glycerol.

FIG. 2 is a table demonstrating sensitivity and specificity for the blood concentration of glycerol above critical levels to diagnose cardiac and other major complications and predict an increased length of ICU or IMCU stay.

FIG. 3 is a table demonstrating the timing (defined as hours post reperfusion) of when the blood concentration of glycerol first becomes critical and for how long the blood concentration of glycerol stay above this critical level. It also demonstrates corresponding values for the blood concentration of lactate.

FIG. 4a-d show graphs comparing Coronary Sinus and systemic values for the blood concentration of glycerol for patients representing four types of clinical course. DCC insert denotes preoperative levels. XC denotes levels at the time of reperfusion followed by the levels at 4, 8, 12, 16, 20 and 24h post reperfusion respectively.

FIG. 5 is a table showing mean values of peak blood concentration of lactate and glycerol for patients with PMI, other complications and without complications, respectively.

### DETAILED AND EXEMPLIFYING DESCRIPTION OF THE INVENTION

A multicenter study of patients undergoing cardiac surgery is demonstrated in this section.

77 Patients undergoing cardiac surgery were monitored peri-operatively using a percutaneously placed intravenous microdialysis catheter (Dipylon Cardiac Catheter, CMA Microdialysis, Solna, Sweden) and positioned in the Coronary Sinus (CS). Coronary lactate and glycerol were obtained hourly for 24-hours post-operatively. In addition, systemic blood concentration of lactate was obtained hourly through an arterial blood gas. Systemic CK-MB and Troponin T (TnT) values were obtained every 4 hours from an arterial blood sample. PMI was defined by CKMB >70 U/L and Troponin T >1.0 μg/L. Other post-operative complications, such as stroke and post-operative bleeding were also noted.

The concentration of glycerol from the Coronary Sinus was analysed in a microdialysis analyser (CMA 600 or ISCUS, CMA Microdialysis, Solna, Sweden), in which glycerol was measured enzymatically.

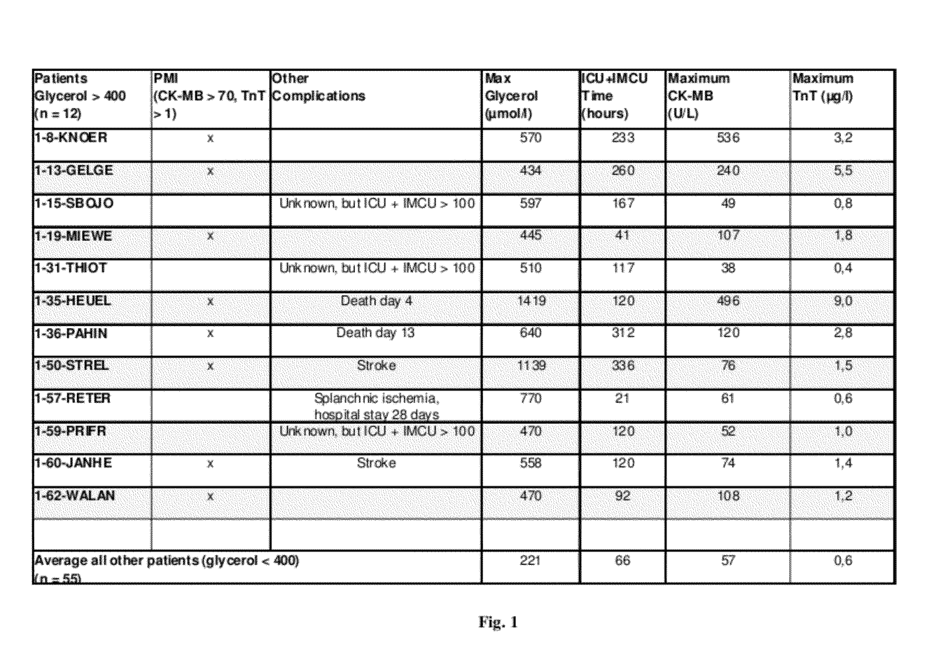

In total, 12 of the 77 patients had values for the blood concentration of glycerol above 400 μmol/l. Of these 12 patients 8 experienced a PMI. The additional 4 patients all had some kind of serious complication, resulting in significantly prolonged time spent in the intensive care unit (ICU), intermediate care unit (IMCU) and/or increased hospital stay. These results are shown in the table of FIG. 1 with comparing values of CKMB and Troponin-T.

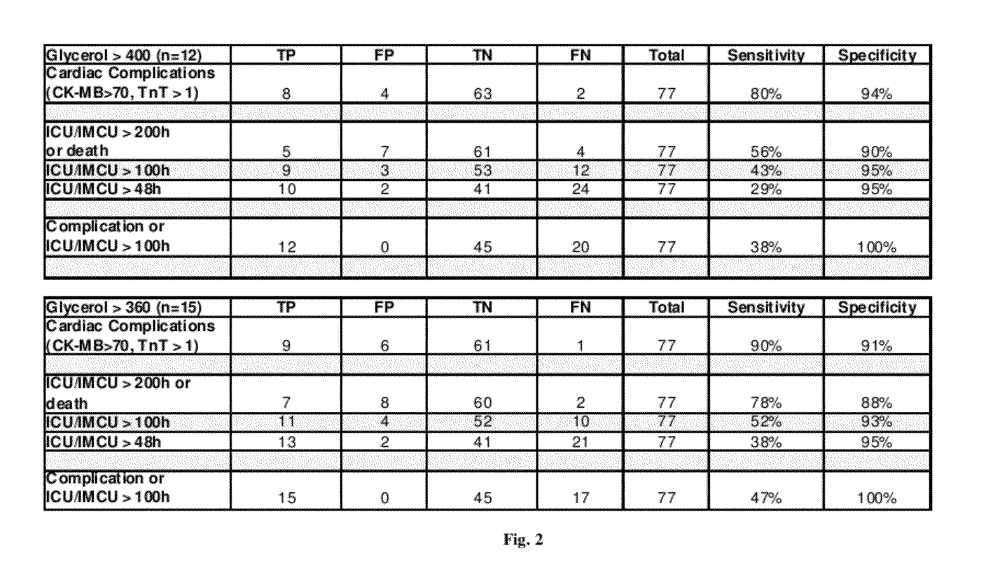

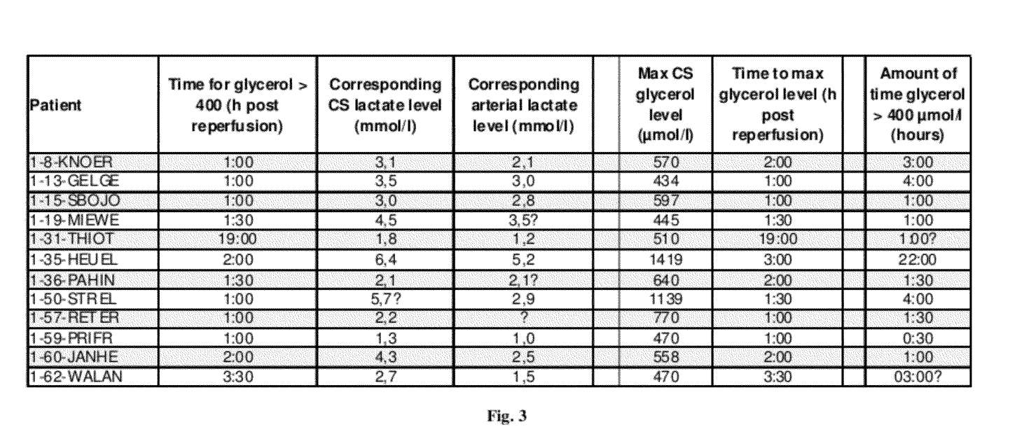

The results shown in FIG. 3 demonstrate that the blood concentration of glycerol increase rapidly at the beginning of an ischemic insult. Most likely, the glycerol levels increase before the ischemic insult has created irreversible damage. This is in contrast to many existing methods to detect major ischemic complications, which become conclusive only after the ischemic damage has become irreversible. Measuring blood glycerol levels in the early post-operative phase would thus greatly enhance the possibilities of active intervention and treatment to prevent the development of irreversible ischemic damage. Further, the results in the table of FIG. 3 indicate that sampling of glycerol should be performed for the first 4 hours. Sampling should be started approximately 30 minutes after reperfusion, when a majority of the glycerol accumulated during the cardiac arrest has been washed out. The results in tables of FIGS. 1 and 2 demonstrate that blood concentration of glycerol above 350-400 μmol/l signal an ongoing serious complication and an expected ICU or IMCU stay above 100 hours, which is 3-4 times the median after cardiac surgery. FIG. 2 further discriminates between true and false positives (TP, FP) and true and false negatives (TN, FN) and determines sensitivity and specificity of the study results according to standard procedures for two different glycerol value thresholds (360 and 400 μmol/).

The results in the table of FIG. 3 further demonstrate that cardiac or other major ischemic complications lead to elevated values for the blood concentration of glycerol for at least one hour. For this reason, sampling of glycerol should be done at least once per hour, to avoid missing a critical value. Further, FIG. 3 shows that the blood concentration of lactate in general increase to >2 mmol/l before or at the same time as the blood concentration of glycerol increases to critical levels.

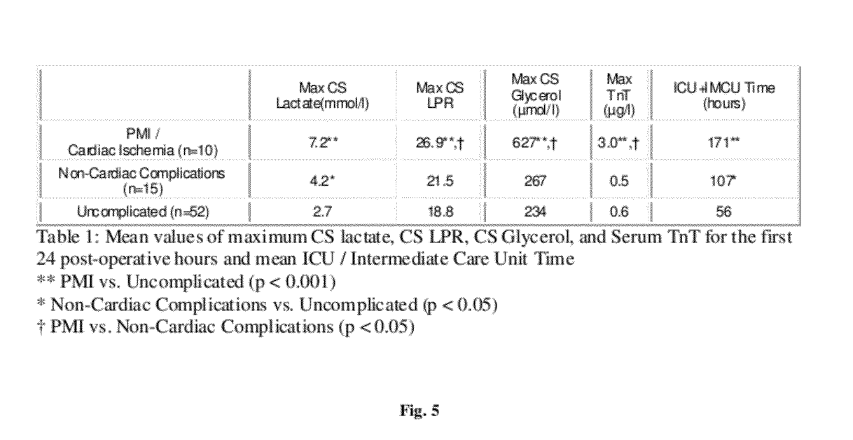

FIG. 5 shows that by combining the evaluation of the blood concentration of glycerol and lactate it becomes possible to differentiate between patients with major ischemic complications and other types of complications and patients without any complication. Patients with major ischemic complications like PMI have significantly elevated blood concentrations of both lactate and glycerol. These patients have a poor clinical outcome and a significantly increased ICU/IMCU stay (3-4 times the average of uncomplicated patients). Patients with other types of complications (in this example mainly post-operative bleeding) have a significantly increased blood concentration of lactate, but the blood concentration of glycerol does not become elevated. Patients with these types of complications have an increased length of ICU stay, but only twice the average of uncomplicated patients.

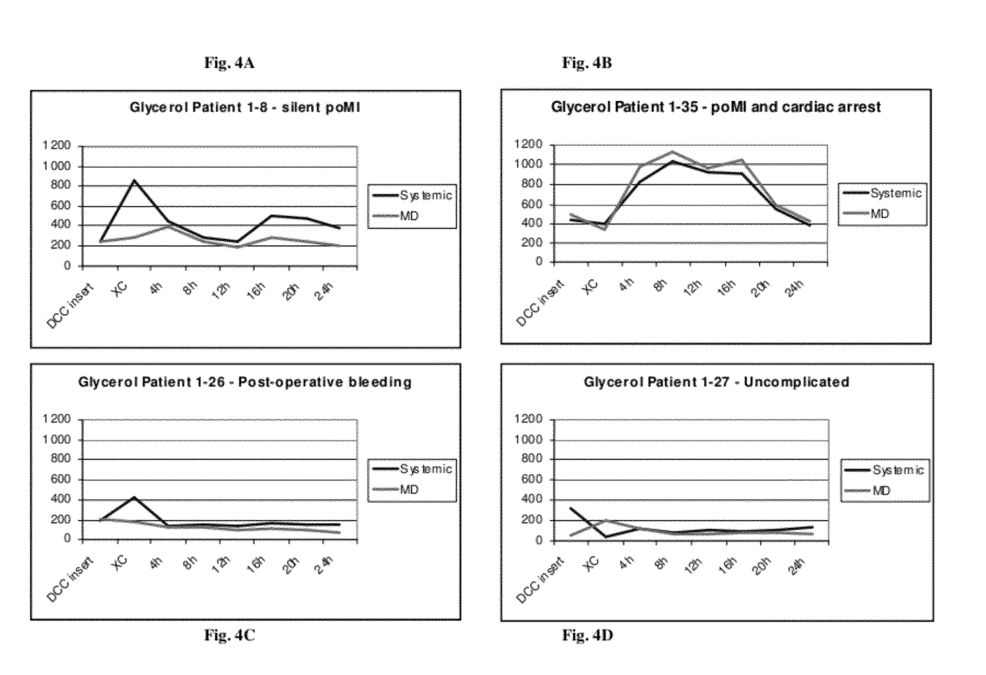

To confirm if the values for the blood concentration of glycerol in the Coronary Sinus were representative of systemic values, a selected number of arterial blood samples were analysed using an automatic luminometric kinetic assay (Hellmér, J. et al, Anal. Biochem. 177, 132-137, 1989), This analysis (see FIG. 4a-d) showed that the systemic and Coronary Sinus blood concentrations are very similar, at least after the immediate reperfusion period. The conclusion is that arterial or central venous sampling of glycerol should be enough to detect complications and that it is not necessary to sample locally in the Coronary Sinus.

In summary, the results of the study show that the blood concentration of glycerol seems to be a highly sensitive and specific marker for early detection of PMI and other major ischemic complications after cardiac surgery and can predict prolonged ICU stay.

1. A method of detecting major ischemic complications in critically ill patients characterized by:
a) determining the blood concentration of glycerol at least once per hour;
b) comparing the so obtained data of the blood concentration of glycerol with a defined critical level of the blood concentration of glycerol; and
c) based on the comparison of step b), identifying patients experiencing or being at risk for major ischemic complications in order to select such patients for intensified surveillance and treatment.

2. A method according to claim 1, based on the comparison of step b), further comprising predicting the length of stay at an intensive care unit.

3. A method according to claim 1, wherein patients identified as experiencing or being at risk for major ischemic complications are patients where the blood concentration of glycerol at least one time has exceeded the above defined critical level of glycerol.

4. A method according to claim 1, wherein patients identified as experiencing or being at risk for major ischemic complications are patients where the blood concentration of glycerol has exceeded the above defined critical level of glycerol during at least one hour.

5. A method according to claim 1, wherein the critical level of glycerol is estimated to be approximately 350 μmol/l to 400 μmol/l.

6. A method according to claim 1, comprising obtaining, from post-surgical patients, data on the blood concentration of glycerol during at least four hours following reperfusion.

7. A method according to claim 6, wherein the patient has undergone cardiac surgery.

8. A method according to claim 6, comprising obtaining data on the blood concentration of glycerol from about 30 minutes after reperfusion.

9. A method according to claim 1, wherein the blood concentration of glycerol is determined enzymatically.

10. A method according to claim 1, further comprising determining the blood concentration of lactate.

11. A method according to claim 10, wherein the blood concentration of glycerol is used in conjunction with the blood concentration of lactate to differentiate between major ischemic complications and other types of complications.

12. A method according to claim 1, wherein the blood concentration of glycerol is the systemic blood concentration.

13. A method according to claim 1, wherein the blood concentration of glycerol is obtained from the venous outflow of an end organ such as the heart, the liver, the kidney or the brain.

14. A method of detecting major ischemic complications, in post-surgical or other critically ill patients characterized by:
a) monitoring the blood concentration of lactate of the patient;
b) detecting an increase in the blood concentration of lactate;
c) determining the blood concentration of glycerol and comparing the so obtained data of the blood concentration of glycerol with a defined critical level of the blood concentration of glycerol; and
d) based on the comparison of step c) identifying patients experiencing or being at risk for major ischemic complications in order to select such patients for intensified surveillance and treatment.

15. A method according to claim 14 based on the comparison of step c) for predicting the length of stay at an intensive care unit.

16. A method according to claim 14, wherein the critical level of glycerol is estimated to be approximately 350 μmol/l to 400 μmol/l.

17. A method according to claim 14, wherein the patient has undergone cardiac surgery.

18. A method according to claim 14, wherein the blood concentration of glycerol is determined enzymatically.

19. A method according to claim 14, wherein the blood concentration of glycerol is the systemic blood concentration.

20. A method according to claim 14, wherein the blood concentration of glycerol is obtained from the venous outflow of an end organ such as the heart, the liver, the kidney or the brain.

21. A method according to claim 14, wherein lactate is measured continuously.
